(19) United States
(12) Patent Application Publication
(10) Pub. No.: US 2014/0357991 A1
(43) Pub. Date: Dec. 4, 2014

(54) Xerostomia Markers

(71) Applicant: Petra Wilder Smith, San Juan Capistrano, CA (US)

(72) Inventor: Petra Wilder Smith, San Juan Capistrano, CA (US)

(21) Appl. No.: 13/905,849

(22) Filed: May 30, 2013

(51) Int. Cl. A61B 5/00

(52) U.S. Cl. CPC A61B 5/4848; A61B 5/0073
USPC 600/427

(57) ABSTRACT

Certain embodiments of the present invention provide methods of determining an efficacy of a xerostomia treatment in a mammalian subject (e.g., a human) that include the steps of comparing a first thickness of a site in the subject that comprised of sub-epithelial oral mucosa to a second thickness of the site; and thereby determining that: the xerostomia treatment is efficacious because the second thickness is greater than the first thickness; or the xerostomia treatment is not efficacious because the first thickness is greater than or equal to the second thickness. In such methods, assaying the first thickness comprises analyzing a first OCT scan of the site that was conducted at a time close to the subject starting the xerostomia treatment, and assaying the second thickness comprises analyzing a second OCT scan of the site that was conducted at a time close to the subject completing the xerostomia treatment.

### PRIORITY DATA

The present application claims the benefit of U.S. Provisional Patent Application Ser. No. 61/653,371, which is hereby incorporated by reference in its entirety and which was filed on May 30, 2012.

### FIELD OF THE INVENTIONS

Embodiments of the inventions relate to markers for xerostomia in a mammalian (e.g., a human) subject. Certain embodiments provide methods for determining the effectiveness of a xerostomia treatment in a subject. In certain embodiments, optical coherence tomography (OCT) images and/or backscatter signal intensity measurements comprise oral mucosa cellular and/or tissue markers in a mammalian (e.g., a human) subject indicative of xerostomia, or a lack, amelioration, or exacerbation thereof in the subject.

### BACKGROUND OF THE INVENTIONS

Xerostomia (also known as dry mouth) is a common oral condition, with an estimated prevalence in 20% to 40% of the population. (Atkinson et al. Dent Clin North Am. 2005; 49:309-326 and Cho et al. Journal of Oral Rehabilitation, 2010; 37(3): 185-193.) Causes for dry mouth include medical condition, salivary gland damage, nerve damage, and drug side effect. Medical conditions associated with dry mouth include diabetes, Sjogren's syndrome, HIV/AIDS, Alzheimer's disease, anemia, cystic fibrosis, rheumatoid arthritis, hypertension, Parkinson's disease, stroke, and mumps. Salivary gland damage from chemotherapy and radiation to the head and neck has been associated with dry mouth, as has nerve damage to the head and neck from injury and surgery. A list of drugs identified as causing dry mouth as a side effect is populated with more than 400 members useful in treating wide-ranging conditions, including depression, anxiety, pain, allergies, colds, obesity, acne, epilepsy, hypertension, diarrhea, nausea, psychotic disorders, urinary incontinence, asthma, and Parkinson's disease. In addition, muscle relaxants, sedatives, and tobacco use have been associated with dry mouth. (Atkinson et al. Dent Clin North Am, 2005; 49:309-326.)

Techniques generally accepted and frequently used for attempting to diagnose and/or evaluate the existence of xerostomia in a subject, or the efficacy of a xerostomia treatment in a subject that has xerostomia, include visual assessment of mucosal appearance, tongue blade adhesion test, saliva collection over a fixed time period, and patient self-evaluation using a standard questionnaire. (Atkinson et al. Dent Clin North Am, 2005; 49:309-326; Cho et al. Journal of Oral Rehabilitation, 2010; 37(3): 185-1931; and Ami et al. Clinical Implant Dentistry and Related Research, 2010; 12:1.) But such techniques are insensitive, subjective, and can require a large patient sample size in order to maximize chances of obtaining useful results in assessing the efficacy of a xerostomia treatment. And such techniques are not sensitive enough to detect certain microstructural characteristics of oral mucosa that exist in a subject suffering from xerostomia and that may: i. abate in as a result of the subject engaging in a candidate xerostomia treatment; or ii. intensify as a result of the subject engaging in a failed xerostomia treatment or other activity. The inability to assess xerostomia (or its amelioration or exacerbation) in an individual subject in a reliable, quantifiable, sensitive, and/or non-subjective manner has been a significant clinical problem and a limiting factor in the development of xerostomia treatments.

### SUMMARY OF THE INVENTIONS

Oral mucosa comprises the mucous membrane epithelium of the mouth. It can be divided into three categories: masticatory mucosa, lining mucosa, and specialized mucosa. Masticatory mucosa, a para-keratinized stratified squamous epithelium, can be found on the dorsum of the tongue, hard palate and attached gingiva. Lining mucosa, a non-keratinized stratified squamous epithelium, can be found almost everywhere else in the oral cavity. Specialized mucosa can be found specifically in the regions of the taste buds on the dorsum of the tongue. Buccal mucosa, a part of the lining mucosa, can be found lining the insides of the cheeks.

Optical coherence tomography (OCT) is a non-invasive and rapid technique capable of generating different types of high resolution images of biological structures, tissues, and cells. OCT can use backscattered light reflected from different layers of biological tissues, structures, cells, and the like to reconstruct high-resolution images thereof. An OCT image can comprise a two-dimensional representation of such optical reflection, and cross-sectional images can be constructed at near histologic resolution (approximately 5-15 μm with current technology). And such images can be stacked to generate 3-D reconstruction images of target tissue, such as epithelial and sub-epithelial structures. OCT is also capable of providing site-specific backscattering data based on coherence-domain optical technology.

Certain embodiments of the present invention provide methods of determining an efficacy of a xerostomia treatment in a mammalian (e.g., a human) subject that include the steps of comparing a first thickness of a site in the subject comprised of epithelial oral mucosa to a second thickness of the site; and determining that: the xerostomia treatment is efficacious because the second thickness is greater than the first thickness; or the xerostomia treatment is not efficacious because the first thickness is greater than or equal to the second thickness. In such methods, identification of the first thickness comprises analyzing data of an OCT scan of the site that was conducted at a time close to the subject starting the xerostomia treatment, and identification of the second thickness comprises analyzing data of an OCT scan of the site that was conducted at a time close to the subject completing the xerostomia treatment.

In some embodiments, the xerostomia treatment is determined efficacious because the second thickness is at least 10% greater than the first thickness. In some embodiments, the xerostomia treatment is determined efficacious because the second thickness is at least 15% greater than the first thickness.

In some embodiments, the site is selected from the group consisting of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

Certain embodiments of the present invention provide methods of determining an efficacy of a xerostomia treatment in a mammalian (e.g., a human) subject that include the steps of comparing a first density of a site in the subject that is comprised of a subepithelial layer of oral mucosa to a second density of the site; and determining that: the xerostomia treatment is efficacious because the second density is greater than the first density; or the xerostomia treatment is not efficacious because the first density is greater than or equal to the second density. In such methods, identification of the first density comprises analyzing data of an OCT scan of the site that was conducted at a time close to the subject starting the xerostomia treatment, and identification of the second density comprises analyzing data of an OCT scan of the site that was conducted at a time close to the subject completing the xerostomia treatment. In some embodiments, the density can comprise a tissue density, a cell density, or a combination thereof of the site.

In some embodiments, the site is selected from the group consisting of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

Certain embodiments of the present invention provide methods of determining an efficacy of a test xerostomia treatment in a mammalian subject (e.g., a human) that include the steps of comparing a first OCT backscatter signal intensity of a site in the subject comprised of an inner-surface of an oral mucosa to a second OCT backscatter signal intensity of the site; and determining that: the xerostomia treatment is efficacious because the first OCT backscatter signal intensity is greater than the second OCT backscatter signal intensity; or the xerostomia treatment is not efficacious because the second OCT backscatter signal intensity is greater than or equal to the first OCT backscatter signal intensity. In such methods, identification of the first OCT backscatter signal intensity comprises analyzing data of an OCT scan of the site that was conducted at a time close to the subject starting the xerostomia treatment; and identification of the second OCT backscatter signal intensity comprises analyzing data of an OCT scan conducted at a time close to the subject completing the xerostomia treatment.

In some embodiments, the xerostomia treatment is determined efficacious because the first OCT backscatter signal intensity is at least 5% greater than the second OCT backscatter signal intensity. In some embodiments, the xerostomia treatment is determined efficacious because the first OCT backscatter signal intensity is at least 10% greater than the second OCT backscatter signal intensity. In some embodiments, the xerostomia treatment is determined efficacious because the first OCT backscatter signal intensity is at least 15% greater than the second OCT backscatter signal intensity.

In some embodiments, the site is selected from the group consisting of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

Certain embodiments of the present invention provide methods of determining an efficacy of a xerostomia treatment in a mammalian subject (e.g., a human) that include the steps of comparing a first ratio and a second ratio; and determining that: the xerostomia treatment is efficacious because the second ratio is less than the first ratio; or the xerostomia treatment is not efficacious because the first ratio is greater than or equal to the second ratio. In such methods, the first ratio can be a first OCT backscatter signal intensity of a first site in the subject comprised of an inner-surface of an oral mucosa: a second OCT backscatter signal intensity of a second site in the subject comprised of a sub-surface of an oral mucosa. The second ratio can be a third OCT backscatter signal intensity of the first site: a fourth OCT backscatter signal intensity of the second site. Identification of each of the first OCT backscatter signal intensity and the second OCT backscatter signal intensity can comprise analyzing data of OCT scans of the first site and the second site, respectively, that were conducted in a period of time close to the subject starting the xerostomia treatment. Identification of each of the third OCT backscatter signal intensity and the fourth OCT backscatter signal intensity can comprise analyzing data of OCT scans of the first site and the second site, respectively, that were conducted in a period of time close to the subject completing the xerostomia treatment.

Certain embodiments of the present invention provide methods of determining an efficacy of a xerostomia treatment in a mammalian subject (e.g., a human) that include the steps of comparing a first ratio and a second ratio; and determining that: the xerostomia treatment is efficacious because the second ratio is less than the first ratio; or the xerostomia treatment is not efficacious because the first ratio is greater than or equal to the second ratio. In such methods, the first ratio can be a first OCT backscatter signal intensity of a first site in the subject comprised of an inner-surface of an oral mucosa: a second OCT backscatter signal intensity of a second site in the subject comprised of an outer-surface of an oral mucosa. The second ratio can be a third OCT backscatter signal intensity of the first site: a fourth OCT backscatter signal intensity of the second site. Identification of each of the first OCT backscatter signal intensity and the second OCT backscatter signal intensity can comprise analyzing data of OCT scans of the first site and the second site, respectively, that were conducted in a period of time close to the subject starting the xerostomia treatment. Identification of each of the third OCT backscatter signal intensity and the fourth OCT backscatter signal intensity can comprise analyzing data of OCT scans of the first site and the second site, respectively, that were conducted in a period of time close to the subject completing the xerostomia treatment.

### BRIEF DESCRIPTION OF THE DRAWINGS

FIGS. 1A-1D show OCT images of a right buccal mucosa of a human subject, approximately 1 cm anterior to the opening of the parotid duct and in the parotid papilla, at various time points of the xerostomia treatment clinical trial described in the Examples. FIG. 1A shows the buccal mucosa of the subject prior to starting (i.e., baseline time point) the clinical trial. FIG. 1B shows an OCT image of the buccal mucosa of the subject after 15 days of brushing teeth with the regular fluoride toothpaste (placebo) in the clinical trial. FIG. 1C shows an OCT image of the buccal mucosa of the subject after seven days of brushing teeth with the Colgate Cavity Protection toothpaste (washout) in the clinical trial. FIG. 1D shows an OCT image of the buccal mucosa of the subject after 15 days of brushing teeth with the dry mouth toothpaste (test) in the clinical trial.

FIGS. 2A-2D are log plots of OCT backscatter signal intensity as a function of tissue depth of a right buccal mucosa of a human subject, approximately 1 cm anterior to the opening of the parotid duct and in the parotid papilla, at various time points of the xerostomia treatment clinical trial described in the Examples. FIG. 2A shows a log plot of OCT backscatter signal intensity as a function of tissue depth of the buccal mucosa of the subject prior to starting (i.e., baseline time point) the clinical trial. FIG. 2B shows a log plot of OCT backscatter signal intensity as a function of tissue depth of the buccal mucosa of the subject after 15 days of brushing teeth with the dry mouth toothpaste (test) in the clinical trial. FIG. 2C shows a log plot of OCT backscatter signal intensity as a function of tissue depth of the buccal mucosa of the subject after seven days of brushing teeth with Colgate Cavity Protection toothpaste (washout) in the clinical trial. FIG. 2D shows a log plot of OCT backscatter signal intensity (y-axis) as a function of tissue depth (x-axis) of the buccal mucosa of the subject after 15 days of brushing teeth the regular fluoride toothpaste (placebo) in the clinical trial.

### DETAILED DESCRIPTION OF THE INVENTIONS

Certain embodiments of the present invention provide markers for xerostomia amelioration or exacerbation in a mammalian (e.g., human) subject undergoing a treatment for xerostomia, which can be a test or an experimental treatment for xerostomia. In some embodiments, markers for xerostomia amelioration comprise a more consistent surface keratinized layer of oral mucosal epithelium (e.g., more consistent in regards to thickness, stratification, integrity, and uniformity of the surface keratinized layer), a thicker epithelial layer of oral mucosa, a denser sub-epithelial layer of oral mucosa, or a combination thereof in a subject after engaging in a treatment for xerostomia or a test treatment for xerostomia. In some embodiments, markers for xerostomia exacerbation comprise a less consistent surface keratinized layer of oral mucosal epithelium (e.g., more consistent in regards to thickness, stratification, integrity, and uniformity of the surface keratinized layer), a thinner epithelial layer of oral mucosa, a less dense sub-epithelial layer of oral mucosa, or a combination thereof in a subject after engaging in a treatment for xerostomia or a test treatment for xerostomia.

In some embodiments, markers for xerostomia amelioration comprise a decrease in an OCT backscatter signal intensity from an inner-surface of an oral mucosa; a decrease in an OCT backscatter signal intensity from a sub-surface of an oral mucosa; a decrease in a ratio of an OCT backscatter signal intensity from an inner-surface of an oral mucosa: an OCT backscatter signal intensity from a sub-surface of an oral mucosa; a decrease in a ratio of an OCT backscatter signal intensity from an inner-surface of an oral mucosa: an OCT backscatter signal intensity from an outer-surface of an oral mucosa; or a combination thereof in a subject (e.g., a human), after the subject engaging in a treatment for xerostomia or a test treatment for xerostomia.

In some embodiments, markers for xerostomia exacerbation comprise an increase in an OCT backscatter signal intensity from an inner-surface of an oral mucosa; an increase in an OCT backscatter signal intensity from a sub-surface of an oral mucosal; an increase in a ratio of an OCT backscatter signal intensity from an inner-surface of an oral mucosa: an OCT backscatter signal intensity from a sub-surface of an oral mucosa; an increase in a ratio of an OCT backscatter signal intensity from an inner-surface of an oral mucosa: an OCT backscatter signal intensity from an outer-surface of an oral mucosa; or a combination thereof in a subject (e.g., a human), after the subject engaging in a treatment for xerostomia or a test treatment for xerostomia.

As used herein, the terms “outer-surface of an oral mucosa,” “inner-surface of an oral mucosa,” and “subsurface of an oral mucosa” include regions of an oral mucosa of a subject having depths ranging, in different areas of the mouth, from approximately 0 to 450 μM, 300 to 900 μM, and 500 to 2000 μM, respectively.

In some embodiments, oral mucosa site(s) comprising markers for xerostomia amelioration or exacerbation include a buccal mucosa, a floor of a mouth, a tongue, a vermillion border of a lower lip, a palate, or a combination thereof in a mammalian (e.g., human) subject.

Techniques useful for determining, in a subject, the consistency of a surface keratinized layer of oral mucosal epithelium, thickness of an epithelial layer of oral mucosa, and/or density of a sub-epithelial layer of oral mucosa include OCT scanning, OCT imaging, and OCT backscatter signal intensity measurements.

In certain embodiments of the invention, a xerostomia treatment, which can be an experimental or a test xerostomia treatment, can comprise administration of a pharmaceutical agent, such as a small molecule, macromolecule, biologic molecule, organic molecule, inorganic molecule, polymer, protein, antibody, RNA sequence, DNA sequence, or combination thereof, or a formulation of such a pharmaceutical agent, to a mammalian (e.g., a human) subject via any effective route, including topical, oral, parental (e.g., infusion, injection, and/or implantation), etc. In some embodiments, a xerostomia treatment or a test xerostomia treatment can comprise a mammalian (e.g., a human) subject engaging in an activity, such as eating a particular diet, wearing a medical device, undergoing psychotherapy, undergoing a surgical procedure, and the like.

The following examples describe and/or illustrate, without limitation, embodiments and/or aspects of the present invention.

### Example 1

### Xerostomia Treatment Clinical Trial Protocol Description

This research study was executed in full compliance University of California, Irvine Institutional Review Board approval #2002-2805. Informed consent was obtained from all subjects prior to their enrollment in the study. Ten subjects previously diagnosed with moderate to severe dry mouth (seven female, three male; ages 37-53, with a mean age of 48) were enrolled in this randomized, double-blind, crossover, placebo-controlled study. Two identically packaged dentifrice products were coded for content and tested. The first dentifrice product was a dry mouth toothpaste (test toothpaste) and the second dentifrice product was a regular, fluoride toothpaste (placebo toothpaste).

In the study, a baseline clinical examination conducted at the beginning of the clinical trial included a tongue blade adhesion test, a visual clinical evaluation, a dry mouth self-evaluation, oral mucosa photographs, and OCT imaging. The tongue blade clinical evaluation, dry mouth self-evaluation, oral mucosa photographs, and OCT imaging and backscatter signal intensity measurements are described below in Examples 2-5 were performed on all enrolled subjects. After the baseline clinical exam, some subjects brushed their teeth with the first dentifrice product for 15 days; followed by a 7-day wash out period in which the subjects brushed their teeth with Colgate Cavity Protection toothpaste; followed by a 15-day period in which the subjects brushed their teeth with the second dentifrice product. After the baseline clinical exam, the other subjects brushed their teeth with the second dentifrice product for 15 days; followed by a 7-day wash out period in which the subjects brushed their teeth with Colgate Cavity Protection toothpaste (washout toothpaste); followed by a 15-day period in which the subjects brushed their teeth with the first dentifrice product. Clinical exams comprising a tongue blade adhesion evaluation, a clinical evaluation, a dry mouth self-evaluation, oral mucosa photographs, OCT scans, and a visual exam, for appearance of mucosal surface moistness versus dryness, desiccation or cracking of mucosal surfaces as well as co-adhesion of dry mucosal surfaces, were acquired at 5-day intervals during use of the first and second dentifrice products, as well as at the end of the 7-day washout period.

Standard toothbrushes and floss were provided to the subjects during the study period; and no other form of oral healthcare (rinses, gels, gum, etc.) was permitted during the study period. Subjects were required to inform the investigators about their use of any new medications that might potentially interfere with the study, including but not limited to antibiotics, antiseptics, decongestants, and antihistamines.

### Example 2

### Tongue Blade Adhesion Clinical Evaluation Description

In each clinical examination conducted during the study, adhesion of a standard, wooden tongue blade to the left and right buccal mucosa, the dorsal and ventral surfaces of the tongue, and the lip was determined by one blinded clinician during each clinical exam conducted in the study. Adhesion was scored as “yes” (score 1) or “no” (score 0) for each site; the lower the cumulative score, the more moist was the mouth, and the higher the score, the drier was the mouth. For each clinical examination conducted in the study, all adhesion scores for an individual subject were added to generate an overall, semi-quantitative tongue blade adhesion score for that subject.

### Example 3

### Dry Mouth Self-Evaluation Questionnaire Description

In each clinical examination conducted in the study, subjects provided responses to a standard dry mouth questionnaire described in Ami et al. Clinical Implant Dentistry and Related Research, 2010; 12:1, the content of which is hereby incorporated by reference in its entirety. The dry mouth questionnaire surveyed, on a scale of 0 to 5, the severity of several dry mouth symptoms potentially experienced by the subjects, with a score of 5 representing the most severe perception of a specific symptom surveyed in the questionnaire and a score of 0 representing the absence of the symptom. For each clinical examination conducted in the study, the scores, of dry mouth symptom surveyed in the questionnaire, provided by an individual subject were added to generate an overall, semi-quantitative dry mouth score for that subject.

### Example 4

### Oral Mucosa Photograph Description

In each clinical examination conducted during the study, oral mucosa photographs of the subjects were taken at each of the OCT imaging locations using a hand-held Nikon camera with ring flash. The photographs were taken for the purposes of determining any visible change in tissue appearance including color, wetness, and texture.

### Example 5

### OCT Scan Description

In each clinical examination conducted during the study, OCT scans were performed on subjects a Niris® OCT console and a flexible fiberoptic imaging probe (Imalux Corporation, Cleveland, Ohio, USA), which allows real-time video rate imaging speed, simultaneous OCT and CCD imaging channels, 3D volumetric imaging, and surface profiling capability at an imaging depth of up to 50 μm, and OCT backscatter signal intensity measurements up to 2 mm. The OCT imaging system has approximately 8 to 15 μm depth resolution and 20 μm lateral resolution. OCT scans for each subject were acquired at 11 oral sites: left and right buccal mucosa, left and right floor of mouth, left and right side of tongue approximately 3 cm from tip, dorsal, and ventral surfaces of tongue approximately 3 cm from tip, left and right vermillion border of lower lip half way between center and angle of mouth, and highest point of palate. Each OCT scan was acquired in less than 1 second and produced images almost immediately.

Prior to any OCT procedure being carried out for a subject, the flexible fiberoptic probe was disinfected by immersion in CIDEX and then covered with a new, sterile probe sheath. Also prior to an OCT procedure being carried out, photographic images of the subject's oral cavity were recorded. During an OCT procedure, scan lines were marked on the recorded photographic images with Photoshop®, to ensure accurate re-localization of OCT scans at subsequent imaging sessions for each subject.

OCT scans were taken with 1310 nm light. OCT backscatter signal intensity comprised a numerical value, calibrated to a known baseline using a pre-standardized phantom with known backscattering properties and reported in arbitrary units (AU).

### Example 6

### OCT Data Description

From each OCT scan conducted on a subject, OCT images were constructed that showed one or more of an epithelial thicknesses, an epithelial density, and an epithelial surface keratinization for oral mucosa at four landmark locations, by superimposing a grid on the OCT images comprised of squares at 1 mm intervals across the width of the scan. The four landmark locations were individualized to each subject in the study, and comprised readily identifiable structural features or characteristics of the subjects' tissue, such as protuberances, indentations, irregularities, or voids. OCT backscatter signal intensity for each of the four landmark locations was also determined from each OCT procedure by using Imalux® system software, to quantify OCT backscatter signal intensity at 1 pixel increments. 2D OCT scans were made at each landmark location; scan length was 1 mm; scan sites were marked on printed out photos of the mucosa to ensure accurate re-localization at successive imaging events

### Example 7

### Analysis Description

The predefined primary efficacy variable of the study was the change in thickness of the epithelium at the four landmark locations for each OCT image site, at each time point. A secondary variable for analysis was the OCT backscatter signal intensity at the epithelial surface and at depth intervals of 1 pixel down to 2 mm, measured at the four landmark locations. For oral mucosa epithelial thickness measurements, OCT image data for each subject were averaged across all scan sites: i.e., left and right buccal mucosa, left and right floor of mouth, left and right side of tongue approximately 3 cm from tip, dorsal, and ventral surfaces of tongue approximately 3 cm from tip, left and right vermillion border of lower lip half way between center and angle of mouth, and highest point of palate. For OCT backscatter signal measurements, OCT backscatter signal intensity as a function of depth were averaged across all scan sites: i.e., left and right buccal mucosa, left and right floor of mouth, left and right side of tongue approximately 3 cm from tip, dorsal, and ventral surfaces of tongue approximately 3 cm from tip, left and right vermillion border of lower lip half way between center and angle of mouth, and highest point of palate.

Data were compared using repeated measures ANOVA with one within-group factor (before vs. after) and one between-group factor (treatment). After adjusting for any between-subject differences, the significance of the interaction factor for time by treatment (F-test with 1 df) provided a test for differences over time between the active and placebo groups.

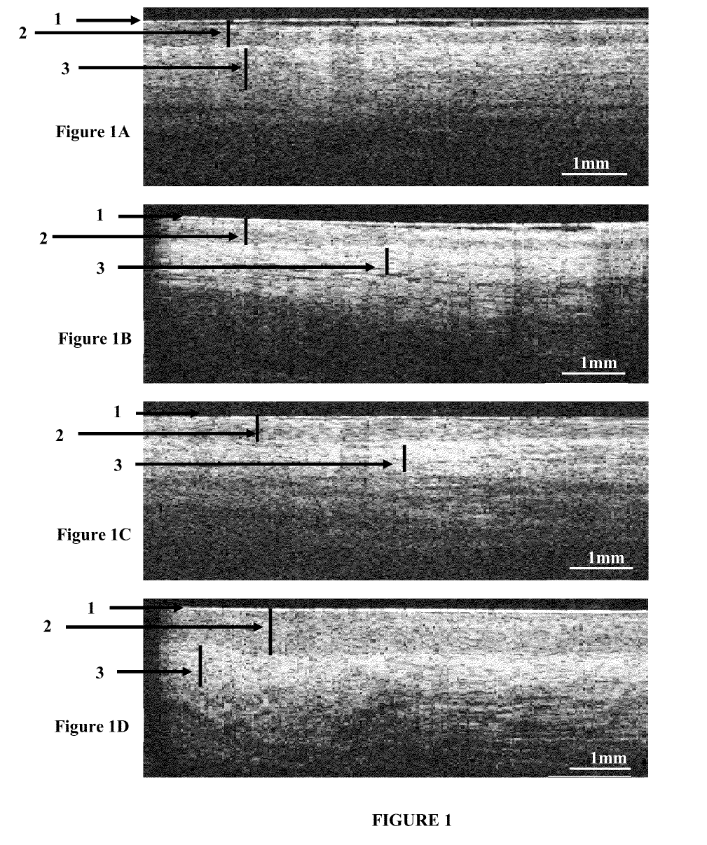

FIGS. 1A-1D show OCT images of a right buccal mucosa of Subject 3, approximately 1 cm anterior to the opening of the parotid duct and in the parotid papilla, at various time points of the xerostomia treatment clinical trial. FIG. 1A shows the surface keratinized layer of epithelium (1), the epithelium layer (2), and the subepithelial layer (3) of the buccal mucosa of the subject prior to starting (i.e., baseline time point) the clinical trial. FIG. 1B shows an OCT image of the surface keratinized layer of epithelium (1), the epithelium layer (2), and the subepithelial layer (3) of the buccal mucosa of the subject after 15 days of brushing teeth with the regular fluoride toothpaste (placebo) in the clinical trial. FIG. 1C shows an OCT image of the surface keratinized layer of epithelium (1), the epithelium layer (2), and the subepithelial layer (3) of the buccal mucosa of the subject after seven days of brushing teeth with the Colgate Cavity Protection toothpaste (washout) in the clinical trial. FIG. 1D shows an OCT image of the surface keratinized layer of epithelium (1), the epithelium layer (2), and the subepithelial layer (3) of the buccal mucosa of the subject after 15 days of brushing teeth with the dry mouth toothpaste (test) in the clinical trial. Use of the placebo product and regular Colgate toothpaste had no measurable effect. The use of the active dry mouth product resulted in significantly improved epithelial surface integrity, significantly increased epithelial and subepithelial thicknesses, and significantly increased epithelial and subepithelial densities.

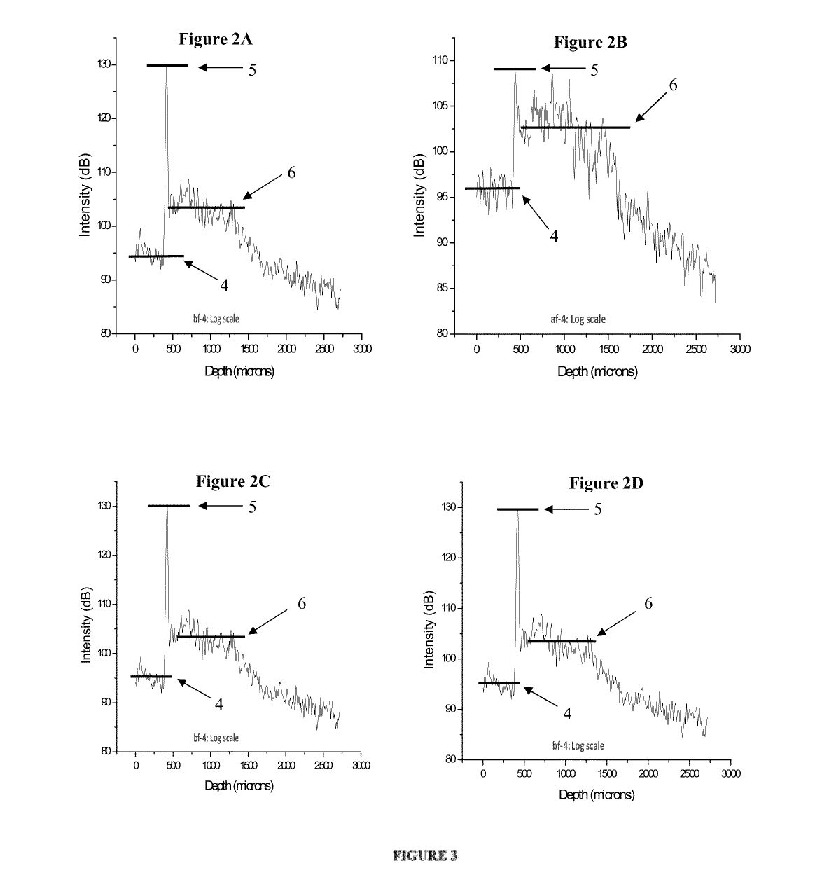

FIGS. 2A-2D are log plots of OCT backscatter signal intensity as a function of tissue depth of a right buccal mucosa of Subject 7, approximately 1 cm anterior to the opening of the parotid duct and in the parotid papilla, at various time points of the xerostomia treatment clinical trial described in the Examples. FIG. 2A shows a log plot of OCT backscatter signal intensity from air above the sample site (4), OCT backscatter signal intensity from the sample site surface (5), and OCT backscatter signal intensity from the immediate subsurface of the sample site (6) of the subject prior to starting (i.e., baseline time point) the clinical trial. Here, (5)/(4)=1.37; (6)/(4)=1.06; and (5)/(6)=1.26. FIG. 2B shows a log plot of OCT backscatter signal intensity from air above the sample site (4), OCT backscatter signal intensity from the sample site surface (5), and OCT backscatter signal intensity from the immediate subsurface of the sample site (6) of the subject after 15 days of brushing teeth with the dry mouth toothpaste (test) in the clinical trial. Here, (5)/(4)=1.05; (6)/(4)=1.07; and (5)/(6)=1.03. FIG. 2C shows a log plot of OCT backscatter signal intensity from air above the sample site (4), OCT backscatter signal intensity from the sample site surface (5), and OCT backscatter signal intensity from the immediate subsurface of the sample site (6) of the subject after seven days of brushing teeth with the Colgate Cavity Protection toothpaste (washout) in the clinical trial. Here, (5)/(4)=1.33; (6)/(4)=1.04; and (5)/(6)=1.22. FIG. 2D shows a log plot of OCT backscatter signal intensity from air above the sample site (4), OCT backscatter signal intensity from the sample site surface (5), and OCT backscatter signal intensity from the immediate subsurface of the sample site (6) of the subject after 15 days of brushing teeth with the regular fluoride toothpaste (placebo) in the clinical trial. Here, (5)/(4)=1.37; (6)/(4)=1.06; and (5)/(6)=1.26. Use of the placebo product and regular Colgate toothpaste had no measurable effect. The use of active dry mouth product resulted in significantly OCT backscatter signal intensities. Treatment related changes were confined to the most superficial 750-1000 μm of the oral mucosa.

In the clinical trial, the mean epithelial thickness and imaging sites combined was 210 μM at baseline (moderate to severe dry mouth), 245 μM after 15 days brushing with placebo, 255 μM after 7 days brushing with washout, and 395 μM after 15 days brushing with dry mouth toothpaste.

### Example 8

### Tongue Blade Adhesion Clinical Evaluation Results

The tongue blade adhesion clinical evaluations of the study did not detect changes in oral dryness in a meaningful way. Improvements in dryness were detected in 4/10 subjects after use of the dry mouth toothpaste and in 5/10 subjects after use of the placebo and/or washout. Thus upon evaluation of a sample size of ten patients, tongue blade adhesion measurements were not sufficiently consistent to be useful in evaluating the dry mouth intervention of the study. The overall tongue blade adhesion scores for each subject at each clinical exam conducted during the study are reported in Table 1.

TABLE 1

| | Overall Tongue Blade Adhesion Score | | | | | | | | | |
|---|---|---|---|---|---|---|---|---|---|---|
| Clinical Exam | S1 | S2 | S3 | S4 | S5 | S6 | S7 | S8 | S9 | S10 |
| Baseline | 5 | 4 | 3 | 3 | 4 | 3 | 5 | 4 | 3 | 5 |
| 1^{st} 5-day TT | 4 | 3 | 4 | 1 | 3 | 3 | 5 | 4 | 5 | 5 |
| brushing period | | | | | | | | | | |
| 2^{nd} 5-day TT | 3 | 2 | 2 | 5 | 3 | 3 | 5 | 4 | 4 | 3 |
| brushing period | | | | | | | | | | |
| 3^{rd} 5-day TT | 2 | 5 | 3 | 4 | 2 | 5 | 5 | 3 | 3 | 3 |
| brushing period | | | | | | | | | | |
| End of washout period | 3 | 5 | 2 | 4 | 3 | 2 | 5 | 3 | 3 | 4 |
| clinical exam | | | | | | | | | | |
| 1^{st} 5-day PT | 4 | 5 | 3 | 4 | 4 | 3 | 4 | 4 | 4 | 3 |
| brushing period | | | | | | | | | | |

“S” = Subject;
“TT” = Test Toothpaste;
“PT” = Placebo Toothpaste

### Example 9

### Dry Mouth Self-Evaluation Questionnaire Results

Dry mouth questionnaire responses among the ten subjects were not consistent, and therefore did not provide a meaningful evaluation of patient response to dry mouth treatments. The overall dry mouth scores for each subject based on the dry mouth questionnaire are reported in Table 2.

TABLE 2

| | Overall Dry Mouth Questionnaire Score | | | | | | | | | |
|---|---|---|---|---|---|---|---|---|---|---|
| Clinical Exam | S1 | S2 | S3 | S4 | S5 | S6 | S7 | S8 | S9 | S10 |
| Baseline | 4 | 5 | 3 | 4 | 5 | 5 | 3 | 5 | 3 | 4 |
| l^{st} 5-day TT | 4 | 4 | 1 | 4 | 5 | 4 | 4 | 5 | 3 | 3 |
| brushing period | | | | | | | | | | |
| 2^{nd} 5-day TT | 4 | 5 | 3 | 5 | 5 | 5 | 4 | 4 | 4 | 3 |
| brushing period | | | | | | | | | | |
| 3^{rd} 5-day TT | 4 | 5 | 3 | 3 | 2 | 4 | 3 | 4 | 4 | 2 |
| brushing period | | | | | | | | | | |
| End of washout period | 4 | 3 | 2 | 4 | 1 | 5 | 5 | 3 | 4 | 2 |
| clinical exam | | | | | | | | | | |
| 1^{st} 5-day PT | 5 | 5 | 3 | 4 | 1 | 3 | 4 | 3 | 5 | 2 |
| brushing period | | | | | | | | | | |
| 2^{nd} 5-day PT | 5 | 4 | 2 | 2 | 3 | 5 | 3 | 3 | 5 | 1 |
| brushing period | | | | | | | | | | |

“S” = Subject;
“TT” = Test Toothpaste;
“PT” = Placebo Toothpaste

### Example 10

### OCT Results

Based on the OCT images of the study, average oral mucosa epithelial thickness increased significantly (p<0.05) after use of the test/dry mouth toothpaste for 15 days, but did not change significantly (p>0.05) after use of the placebo/regular fluoride toothpaste or washout/Colgate Cavity Protection toothpaste. The average oral mucosa epithelial thicknesses measured for each subject at each clinical exam conducted during the study are reported in the following Tables. The sites at which the oral mucosal epithelial thicknesses reported in the following Tables were taken were: left buccal mucosa (L1); right buccal mucosa (L2), left floor of mouth (L3); right floor of mouth (LA), left side of tongue approximately 3 cm from tip (L5); right side of tongue approximately 3 cm from tip (Lb), dorsal surface of tongue approximately 3 cm from tip (L7); ventral surface of tongue approximately 3 cm from tip (L8); left vermillion border of lower lip half way between center and angle of mouth (L9); right vermillion border of lower lip half way between center and angle of mouth (L10); and highest point of palate (L11).

TABLE 3
(Subject 1)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 205 | 195 | 180 | 225 | 250 | 235 | 260 | 180 | 190 | 200 | 200 |
| 1^{st} 5-day TT | 260 | 250 | 245 | 290 | 300 | 290 | 355 | 260 | 235 | 250 | 210 |
| brushing period | | | | | | | | | | | |
| 2^{nd} 5-day TT | 340 | 345 | 305 | 365 | 425 | 345 | 450 | 285 | 280 | 290 | 240 |
| brushing period | | | | | | | | | | | |
| 3^{rd} 5-day TT | 380 | 365 | 370 | 450 | 470 | 440 | 460 | 300 | 295 | 310 | 305 |
| brushing period | | | | | | | | | | | |
| End of washout | 215 | 220 | 200 | 240 | 295 | 250 | 275 | 190 | 205 | 200 | 225 |
| period | | | | | | | | | | | |
| clinical exam | | | | | | | | | | | |
| 1^{st} 5-day PT | 215 | 205 | 240 | 205 | 280 | 250 | 270 | 190 | 205 | 200 | 210 |
| brushing period | | | | | | | | | | | |
| 2^{nd} 5-day PT | 220 | 215 | 225 | 235 | 255 | 250 | 285 | 190 | 215 | 200 | 220 |
| brushing period | | | | | | | | | | | |
| 3^{rd} 5-day PT | 210 | 210 | 225 | 235 | 250 | 250 | 285 | 195 | 210 | 200 | 220 |
| brushing period | | | | | | | | | | | |

“TT” = Test Toothpaste;
“PT” = Placebo Toothpaste

TABLE 4
(Subject 2)

| | OCT Average Oral Mucosa Epithelial Thickness | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 215 | 185 | 180 | 235 | 250 | 235 | 260 | 185 | 190 | 220 | 210 |
| 1^{st} 5-day TT | 250 | 245 | 245 | 300 | 310 | 280 | 355 | 260 | 240 | 215 | 250 |
| brushing period | | | | | | | | | | | |
| 2^{nd} 5-day TT | 350 | 305 | 305 | 375 | 415 | 345 | 390 | 285 | 280 | 290 | 240 |
| brushing period | | | | | | | | | | | |
| 3^{rd} 5-day TT | 360 | 335 | 370 | 470 | 460 | 450 | 470 | 310 | 300 | 320 | 305 |
| brushing period | | | | | | | | | | | |
| End of washout | 225 | 215 | 200 | 245 | 235 | 300 | 270 | 190 | 275 | 200 | 205 |
| period | | | | | | | | | | | |
| clinical exam | | | | | | | | | | | |
| 1^{st} 5-day PT | 215 | 215 | 240 | 225 | 275 | 260 | 270 | 190 | 215 | 185 | 210 |
| brushing period | | | | | | | | | | | |
| 2^{nd} 5-day PT | 225 | 215 | 225 | 235 | 265 | 255 | 285 | 195 | 210 | 200 | 220 |
| brushing period | | | | | | | | | | | |
| 3^{rd} 5-day PT | 215 | 215 | 225 | 215 | 260 | 260 | 275 | 190 | 215 | 200 | 225 |
| brushing period | | | | | | | | | | | |

“TT” = Test Toothpaste;
“PT” = Placebo Toothpaste

TABLE 5
(Subject 3)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 225 | 185 | 180 | 235 | 250 | 235 | 260 | 185 | 190 | 220 | 205 |
| 1^{st} 5-day TT | 250 | 265 | 245 | 310 | 310 | 280 | 355 | 260 | 240 | 215 | 250 |
| brushing period | | | | | | | | | | | |
| 2^{nd} 5-day TT | 350 | 305 | 300 | 375 | 415 | 345 | 390 | 285 | 280 | 260 | 240 |
| brushing period | | | | | | | | | | | |
| 3^{rd} 5-day TT | 360 | 335 | 370 | 470 | 470 | 450 | 470 | 310 | 300 | 320 | 305 |
| brushing period | | | | | | | | | | | |
| End of washout | 225 | 215 | 200 | 245 | 235 | 200 | 270 | 190 | 275 | 200 | 205 |
| period | | | | | | | | | | | |
| clinical exam | | | | | | | | | | | |
| 1^{st} 5-day PT | 205 | 215 | 240 | 245 | 275 | 240 | 270 | 190 | 215 | 185 | 210 |
| brushing period | | | | | | | | | | | |
| 2^{nd} 5-day PT | 225 | 220 | 225 | 235 | 265 | 255 | 285 | 195 | 210 | 205 | 220 |
| brushing period | | | | | | | | | | | |
| 3^{rd} 5-day PT | 215 | 215 | 225 | 215 | 280 | 260 | 275 | 190 | 215 | 200 | 210 |
| brushing period | | | | | | | | | | | |

“TT” = Test Toothpaste;
“PT” = Placebo Toothpaste

TABLE 6
(Subject 4)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 275 | 205 | 180 | 245 | 250 | 235 | 280 | 185 | 190 | 220 | 200 |
| 1^{st} 5-day TT | 340 | 265 | 245 | 310 | 310 | 280 | 315 | 260 | 240 | 215 | 200 |
| brushing period | | | | | | | | | | | |
| 2^{nd} 5-day TT | 350 | 310 | 320 | 375 | 415 | 345 | 350 | 285 | 280 | 260 | 230 |
| brushing period | | | | | | | | | | | |
| 3^{rd} 5-day TT | 360 | 340 | 370 | 470 | 470 | 450 | 440 | 310 | 300 | 320 | 305 |
| brushing period | | | | | | | | | | | |
| End of washout | 215 | 225 | 205 | 245 | 235 | 200 | 270 | 190 | 275 | 200 | 205 |
| period | | | | | | | | | | | |
| clinical exam | | | | | | | | | | | |
| 1^{st} 5-day PT | 205 | 245 | 240 | 215 | 275 | 240 | 270 | 200 | 215 | 185 | 210 |
| brushing period | | | | | | | | | | | |
| 2^{nd} 5-day PT | 220 | 250 | 225 | 235 | 265 | 275 | 285 | 195 | 210 | 200 | 190 |
| brushing period | | | | | | | | | | | |
| 3^{rd} 5-day PT | 235 | 245 | 225 | 225 | 280 | 250 | 275 | 190 | 215 | 200 | 220 |
| brushing period | | | | | | | | | | | |

“TT” = Test Toothpaste;
“PT” = Placebo Toothpaste

TABLE 7
(Subject 5)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 200 | 205 | 195 | 240 | 230 | 240 | 300 | 185 | 195 | 220 | 200 |
| 1^{st} 5-day TT | 250 | 265 | 245 | 310 | 310 | 280 | 315 | 260 | 240 | 215 | 200 |
| brushing period | | | | | | | | | | | |
| 2^{nd} 5-day TT | 310 | 310 | 320 | 375 | 415 | 345 | 350 | 285 | 265 | 260 | 230 |
| brushing period | | | | | | | | | | | |
| 3^{rd} 5-day TT | 345 | 340 | 370 | 470 | 470 | 450 | 440 | 310 | 310 | 260 | 325 |
| brushing period | | | | | | | | | | | |
| End of washout | 215 | 225 | 205 | 245 | 235 | 200 | 270 | 190 | 275 | 200 | 205 |
| period | | | | | | | | | | | |
| clinical exam | | | | | | | | | | | |
| 1^{st} 5-day PT | 225 | 245 | 240 | 215 | 275 | 240 | 270 | 200 | 215 | 185 | 230 |
| brushing period | | | | | | | | | | | |
| 2^{nd} 5-day PT | 200 | 250 | 225 | 235 | 265 | 275 | 285 | 195 | 210 | 200 | 195 |
| brushing period | | | | | | | | | | | |
| 3^{rd} 5-day PT | 205 | 245 | 225 | 225 | 280 | 250 | 275 | 190 | 215 | 200 | 200 |
| brushing period | | | | | | | | | | | |

“TT” = Test Toothpaste;
“PT” = Placebo Toothpaste

TABLE 8
(Subject 6)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 200 | 225 | 195 | 240 | 230 | 240 | 300 | 185 | 195 | 220 | 200 |
| 1^{st} 5-day TT | 245 | 250 | 245 | 310 | 310 | 280 | 315 | 260 | 240 | 245 | 200 |
| brushing period | | | | | | | | | | | |
| 2^{nd} 5-day TT | 310 | 295 | 320 | 375 | 415 | 345 | 350 | 285 | 265 | 270 | 230 |
| brushing period | | | | | | | | | | | |
| 3^{rd} 5-day TT | 365 | 340 | 370 | 470 | 470 | 450 | 440 | 310 | 310 | 360 | 315 |
| brushing period | | | | | | | | | | | |
| End of washout | 215 | 235 | 205 | 245 | 235 | 200 | 270 | 190 | 275 | 200 | 205 |
| period | | | | | | | | | | | |
| clinical exam | | | | | | | | | | | |
| 1^{st} 5-day PT | 225 | 245 | 240 | 215 | 275 | 240 | 270 | 200 | 235 | 185 | 230 |
| brushing period | | | | | | | | | | | |
| 2^{nd} 5-day PT | 200 | 250 | 245 | 235 | 265 | 275 | 275 | 195 | 210 | 210 | 195 |
| brushing period | | | | | | | | | | | |
| 3^{rd} 5-day PT | 205 | 295 | 245 | 225 | 270 | 250 | 265 | 190 | 220 | 200 | 205 |
| brushing period | | | | | | | | | | | |

“TT” = Test Toothpaste;
“PT” = Placebo Toothpaste

TABLE 9
(Subject 7)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 205 | 225 | 150 | 220 | 230 | 245 | 300 | 145 | 165 | 240 | 180 |
| l^{st} 5-day TT | 260 | 250 | 195 | 310 | 310 | 280 | 315 | 220 | 200 | 245 | 200 |
| brushing period | | | | | | | | | | | |
| 2^{nd} 5-day TT | 320 | 295 | 260 | 375 | 415 | 345 | 350 | 285 | 265 | 270 | 230 |
| brushing period | | | | | | | | | | | |
| 3^{rd} 5-day TT | 460 | 340 | 310 | 420 | 450 | 450 | 440 | 310 | 310 | 360 | 315 |
| brushing period | | | | | | | | | | | |
| End of washout | 215 | 235 | 165 | 245 | 235 | 230 | 270 | 160 | 275 | 220 | 205 |
| period | | | | | | | | | | | |
| clinical exam | | | | | | | | | | | |
| 1^{st} 5-day PT | 220 | 245 | 165 | 215 | 245 | 230 | 270 | 150 | 235 | 245 | 200 |
| brushing period | | | | | | | | | | | |
| 2^{nd} 5-day PT | 230 | 250 | 165 | 235 | 255 | 235 | 275 | 165 | 210 | 220 | 195 |
| brushing period | | | | | | | | | | | |
| 3^{rd} 5-day PT | 230 | 295 | 165 | 225 | 230 | 230 | 285 | 150 | 220 | 220 | 205 |
| brushing period | | | | | | | | | | | |

“TT” = Test Toothpaste;
“PT” = Placebo Toothpaste

TABLE 10
(Subject 8)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 220 | 205 | 195 | 245 | 230 | 240 | 280 | 185 | 195 | 220 | 200 |
| 1^{st} 5-day TT | 260 | 255 | 205 | 315 | 300 | 280 | 335 | 260 | 240 | 260 | 290 |
| brushing period | | | | | | | | | | | |
| 2^{nd} 5-day TT | 340 | 315 | 270 | 355 | 425 | 345 | 350 | 285 | 265 | 295 | 380 |
| brushing period | | | | | | | | | | | |
| 3^{rd} 5-day TT | 365 | 350 | 270 | 450 | 460 | 390 | 440 | 310 | 310 | 470 | 465 |
| brushing period | | | | | | | | | | | |
| End of washout | 235 | 215 | 215 | 245 | 245 | 200 | 300 | 190 | 275 | 210 | 215 |
| period | | | | | | | | | | | |
| clinical exam | | | | | | | | | | | |
| 1^{st} 5-day PT | 225 | 225 | 200 | 225 | 270 | 240 | 270 | 200 | 215 | 225 | 220 |
| brushing period | | | | | | | | | | | |
| 2^{nd} 5-day PT | 230 | 220 | 205 | 225 | 260 | 275 | 275 | 195 | 210 | 220 | 235 |
| brushing period | | | | | | | | | | | |
| 3^{rd} 5-day PT | 235 | 215 | 205 | 235 | 270 | 250 | 280 | 190 | 215 | 220 | 220 |
| brushing period | | | | | | | | | | | |

“TT” = Test Toothpaste;
“PT” = Placebo Toothpaste

TABLE 11
(Subject 9)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 215 | 225 | 150 | 220 | 230 | 240 | 300 | 195 | 175 | 240 | 180 |
| 1^{st} 5-day TT | 260 | 250 | 195 | 310 | 310 | 280 | 315 | 220 | 200 | 235 | 200 |
| brushing period | | | | | | | | | | | |
| 2^{nd} 5-day TT | 320 | 295 | 260 | 375 | 415 | 345 | 350 | 285 | 265 | 270 | 230 |
| brushing period | | | | | | | | | | | |
| 3^{rd} 5-day TT | 360 | 340 | 310 | 420 | 450 | 450 | 440 | 310 | 310 | 360 | 315 |
| brushing period | | | | | | | | | | | |
| End of washout | 215 | 235 | 165 | 245 | 245 | 230 | 275 | 160 | 275 | 220 | 205 |
| period | | | | | | | | | | | |
| clinical exam | | | | | | | | | | | |
| 1^{st} 5-day PT | 210 | 245 | 165 | 215 | 245 | 230 | 270 | 150 | 235 | 245 | 200 |
| brushing period | | | | | | | | | | | |
| 2^{nd} 5-day PT | 230 | 250 | 155 | 235 | 250 | 235 | 275 | 165 | 210 | 220 | 195 |
| brushing period | | | | | | | | | | | |
| 3^{rd} 5-day PT | 230 | 295 | 170 | 225 | 230 | 230 | 285 | 150 | 220 | 225 | 205 |
| brushing period | | | | | | | | | | | |

“TT” = Test Toothpaste;
“PT” = Placebo Toothpaste

TABLE 12
(Subject 10)

| | OCT Average Oral Mucosa Epithelial Thickness (μM) | | | | | | | | | | |
|---|---|---|---|---|---|---|---|---|---|---|---|
| Clinical Exam | L1 | L2 | L3 | L4 | L5 | L6 | L7 | L8 | L9 | L10 | L11 |
| Baseline | 205 | 210 | 200 | 245 | 235 | 245 | 315 | 200 | 205 | 225 | 205 |
| 1^{st} 5-day TT | 260 | 265 | 245 | 310 | 310 | 280 | 315 | 260 | 240 | 215 | 200 |
| brushing period | | | | | | | | | | | |
| 2^{nd} 5-day TT | 320 | 315 | 320 | 395 | 415 | 345 | 350 | 285 | 265 | 260 | 230 |
| brushing period | | | | | | | | | | | |
| 3^{rd} 5-day TT | 365 | 345 | 390 | 470 | 470 | 450 | 440 | 310 | 320 | 260 | 325 |
| brushing period | | | | | | | | | | | |
| End of washout | 220 | 240 | 205 | 245 | 235 | 200 | 280 | 190 | 235 | 200 | 205 |
| period | | | | | | | | | | | |
| clinical exam | | | | | | | | | | | |
| 1^{st} 5-day PT | 230 | 225 | 240 | 245 | 235 | 240 | 270 | 200 | 215 | 185 | 230 |
| brushing period | | | | | | | | | | | |
| 2^{nd} 5-day PT | 210 | 220 | 225 | 235 | 245 | 275 | 285 | 195 | 220 | 200 | 195 |
| brushing period | | | | | | | | | | | |
| 3^{rd} 5-day PT | 215 | 225 | 195 | 245 | 240 | 270 | 295 | 190 | 235 | 200 | 210 |
| brushing period | | | | | | | | | | | |

“TT” = Test Toothpaste;
“PT” = Placebo Toothpaste

In OCT backscatter signal intensity measurements, there was generally observed an intense backscattered signal at an outer-surface epithelial oral mucosa and OCT backscatter signal intensity becoming progressively less intense with increasing depth into an inner-surface and sub-surface oral mucosa. With use of the test/dry mouth toothpaste, OCT backscatter signal data obtained from OCT procedures conducted on subjects showed progressive and characteristic changes from baseline. An OCT backscatter signal intensity ratio of inner-surface epithelial oral mucosa: outer-surface oral mucosa decreased significantly (p<0.05) after five days use of the test/dry mouth toothpaste, with another significant decrease (p<0.05) after 15 days use. A similar, but somewhat weaker trend was observed for an OCT backscatter signal intensity ratio of inner-surface epithelial oral mucosa: subsurface epithelial oral mucosa. Treatment-related changes in OCT backscatter signal intensity measurements were confined to the most superficial 750-1000 μm of the oral mucosa.

The skilled artisan will recognize the interchangeability of various features from different embodiments. Although the disclosure has been provided in the context of certain embodiments and examples, it will be understood by those skilled in the art that the disclosure extends beyond the specifically described embodiments to other alternative embodiments and/or uses and obvious modifications and equivalents thereof. Accordingly, the disclosure is not intended to be limited by the specific disclosures of embodiments herein.

What is claimed is:

1. A method of determining an efficacy of a xerostomia treatment in a mammalian subject, the method comprising comparing a first thickness of a site in the subject comprised of epithelial oral mucosa to a second thickness of the site; and determining that:
the xerostomia treatment is efficacious because the second thickness is greater than the first thickness; or
the xerostomia treatment is not efficacious because the first thickness is greater than or equal to the second thickness;
wherein an identification of the first thickness comprises analyzing data of a first optical coherence tomography (OCT) scan of the site that was conducted at a time close to the subject starting the xerostomia treatment; and wherein an identification of the second thickness comprises analyzing data of a second OCT scan of the site that was conducted at a time close to the subject completing the xerostomia treatment.

2. A method of determining an efficacy of a xerostomia treatment in a mammalian subject, the method comprising comparing a first density of a site in the subject comprised of a subepithelial layer of oral mucosa to a second density of the site; and determining that:
the xerostomia treatment is efficacious because the second density is greater than the first density; or
the xerostomia treatment is not efficacious because the first density is greater than or equal to the second density,
wherein an identification of the first density comprises analyzing data of a first OCT scan of the site that was conducted at a time close to the subject starting the xerostomia treatment; and
wherein an identification of the second density comprises analyzing data of a second OCT scan of the site that was conducted at a time close to the subject completing the xerostomia treatment.

3. A method of determining an efficacy of a xerostomia treatment in a mammalian subject, the method comprising comparing a first OCT backscatter signal intensity of a site in the subject comprised of an inner surface of an oral mucosa or a subsurface of an oral mucosa to a second determined OCT backscatter signal intensity of the site; and determining that:
the xerostomia treatment is efficacious because the second OCT backscatter signal intensity is less than the first OCT backscatter signal intensity; or
the xerostomia treatment is not efficacious because the second OCT backscatter signal intensity is greater than or the same as the first OCT backscatter signal intensity,
wherein an identification of the first OCT backscatter signal intensity comprises analyzing data of a first OCT scan that was conducted at a time close to the subject starting the xerostomia treatment; and wherein an identification of the second OCT backscatter signal intensity comprises analyzing data of a second OCT scan that was conducted at a time close to the subject completing the xerostomia treatment.

4. The method of claim 1, wherein the subject is a human.

5. The method of claim 2, wherein the subject is a human.

6. The method of claim 3, wherein the subject is a human.

7. The method of claim 4, wherein the site is further characterized by being located at one of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

8. The method of claim 5, wherein the site is further characterized by being located at one of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

9. The method of claim 6, wherein the site is further characterized by being located at one of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

7. The method of claim 4, wherein the xerostomia treatment comprises an experimental xerostomia treatment, and wherein the identification of the first thickness comprises constructing a first OCT image from the data of the first OCT scan, and wherein the identification of the second thickness comprises constructing a second OCT image from the data of the second OCT scan.

8. The method of claim 5, wherein the xerostomia treatment comprises an experimental xerostomia treatment, and wherein the identification of the first density comprises constructing a first OCT image from the data of the first OCT scan, and wherein the identification of the second density comprises constructing a second OCT image from the data of the second OCT scan.

9. The method of claim 6, wherein the xerostomia treatment comprises an experimental xerostomia treatment, and wherein the identification of the first OCT backscatter signal intensity comprises constructing, from the data of the first OCT scan, a first plot of OCT backscatter signal intensity as a function of tissue depth, and wherein the identification of the second OCT backscatter signal intensity comprises constructing, from the data of the second OCT scan, a second plot of OCT backscatter signal intensity as a function of tissue depth.

13. A method of determining an efficacy of a xerostomia treatment in a mammalian subject, the method comprising comparing a first ratio and a second ratio; and determining that:
the xerostomia treatment is efficacious because the second ratio is less than the first ratio; or
the xerostomia treatment is not efficacious because the first ratio is greater than or equal to the second ratio,
wherein the first ratio is a first OCT backscatter signal intensity of a first site in the subject comprised of an inner-surface of an oral mucosa: a second OCT backscatter signal intensity of a second site in the subject comprised of a subsurface of an oral mucosa; and wherein the second ratio is a third OCT backscatter signal intensity of the first site: a fourth OCT backscatter signal intensity of the second site; and wherein identification of each of the first OCT backscatter signal intensity and the second OCT backscatter signal intensity comprise analyzing data of OCT scans of the first site and the second site, respectively, that were each conducted in a period of time close to the subject starting the xerostomia treatment; and wherein identification of each of the third OCT backscatter signal intensity and the fourth OCT backscatter signal intensity comprises analyzing data of OCT scans of the first site and the second site, respectively, that were each conducted in a period of time close to the subject completing the xerostomia treatment.

14. A method of determining an efficacy of a xerostomia treatment in a mammalian subject, the method comprising comparing a first ratio and a second ratio; and determining that:
the xerostomia treatment is efficacious because the second ratio is less than the first ratio; or
the xerostomia treatment is not efficacious because the first ratio is greater than or equal to the second ratio,
wherein the first ratio is a first OCT backscatter signal intensity of a first site in the subject comprised of an inner surface of an oral mucosa: a second OCT backscatter signal intensity of a second site in the subject comprised of an outer surface of an oral mucosa; and wherein the second ratio is a third OCT backscatter signal intensity of the first site: a fourth OCT backscatter signal intensity of the second site; and wherein identification of each of the first OCT backscatter signal intensity and the second OCT backscatter signal intensity comprise analyzing data of OCT scans of the first site and the second site, respectively, that were each conducted in a period of time close to the subject starting the xerostomia treatment; and wherein identification of each of the third OCT backscatter signal intensity and the fourth OCT backscatter signal intensity comprises analyzing data of OCT scans of the first site and the second site, respectively, that were each conducted in a period of time close to the subject completing the xerostomia treatment.

15. The method of claim 13, wherein the subject is a human.

16. The method of claim 14, wherein the subject is a human.

17. The method of claim 15, wherein each of the first site and the second site are further characterized by being located at one of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

18. The method of claim 16, wherein each of the first site and the second site are further characterized by being located at one of a left buccal mucosa of the subject, a right buccal mucosa of the subject, a left floor of a mouth of the subject, a right floor the mouth of the subject, a left side of a tongue of the subject, a right side of the tongue of the subject, a dorsal surface of the tongue of the subject, a ventral surface of the tongue of the subject, a left vermillion border of a lower lip of the subject, a right vermillion border of the lower lip of the subject, and a highest point of a palate of the subject.

19. The method of claim 15, wherein the xerostomia treatment comprises an experimental xerostomia treatment.

20. The method of claim 16, wherein the xerostomia treatment comprises an experimental xerostomia treatment.
